(19) United States
(12) Patent Application Publication
(10) Pub. No.: US 2024/0240216 A1
(43) Pub. Date: Jul. 18, 2024

(54) MATERIAL AND METHOD FOR PREPARATION OF SMALL, SINGLE-STRANDED DNA CIRCLES

(71) Applicant: Purdue Research Foundation, West Lafayette, IN (US)

(72) Inventors: Chengde Mao, West Lafayette, IN (US); Victoria Elizabeth Paluzzi, Boise, ID (US); Cuizheng Zhang, West Lafayette, IN (US)

(73) Assignee: Purdue Research Foundation, West Lafayette, IN (US)

(21) Appl. No.: 18/411,891

(22) Filed: Jan. 12, 2024

Related U.S. Application Data
(60) Provisional application No. 63/439,006, filed on Jan. 13, 2023.

(51) Int. Cl. C12P 19/34; C12N 9/00

(52) U.S. Cl. CPC C12P 19/34; C12N 9/93; C12Y 605/01002

(57) ABSTRACT

A splint single-stranded DNA (ssDNA), which comprises a linear splint sequence and a hairpin sequence on each side of the linear splint sequence; a splint ssDNA-ligating linear ssDNA monomeric complex; a method of producing a ssDNA circle; and a kit comprising the splint ssDNA and instructions for using the splint ssDNA to produce a ssDNA circle.

### CROSS-REFERENCE TO RELATED APPLICATIONS

This application claims priority to U.S. provisional patent application No. 63/439,006, which was filed on Jan. 13, 2023, and the contents of which are hereby incorporated by reference.

### STATEMENT OF GOVERNMENT SUPPORT

This invention was made with government support under CMMI 2025187 and CCF 2107393 awarded by the National Science Foundation. The government has certain rights in the invention.

### TECHNICAL FIELD

The present disclosure relates to a material and a method for preparing small, single-stranded DNA circles.

### SEQUENCE LISTINGS

The sequences herein (SEQ ID NOS: 1-24) are also provided in computer-readable form encoded in a file filed herewith and incorporated herein by reference. The information recorded in computer-readable form is identical to the written Sequence Listing provided below, pursuant to 37 C.F.R. § 1.821(f).

### BACKGROUND

This section introduces aspects that may help facilitate a better understanding of the disclosure. Accordingly, these statements are to be read in this light and are not to be construed as admissions about what is or is not prior art.

Single-stranded DNA (ssDNA) circles have many technological applications. For example, ssDNA circles are resistant to exonuclease, and cyclization is an efficient way to improve stability of antisense DNAs, thereby addressing a major challenge for application of antisense technology. ssDNA circles also can be used as templates for rolling circle amplification, which is a widely used method for signal amplification, isothermal DNA amplification, capturing microRNAs, and scaffolding DNA nano-construction.

The common method for preparing a ssDNA circle is to use a splint ssDNA to base pair with and bring together the two ends of a ssDNA, then use T4 ligase to join covalently the two ends of the ssDNA. The method works well when the ssDNA is long (i.e., greater than 30 bases); however, when the ssDNA is short, the monomeric ssDNA-splint DNA complex is strained and unstable. Consequently, the ssDNA and splint ssDNA tend to form oligomeric complexes, leading to ssDNA oligomerization upon ligation.

Many efforts have been devoted to overcoming the problem of cyclization of small ssDNA; however, none of them is satisfactory. A special type of ligase has been developed and is commercially available. The enzyme, CircLigase™ (Lucigen Corp.), is very expensive, has poor ligation efficiency, and only works for ssDNA longer than 30 bases. The use of an extremely low ssDNA concentration also has been explored to suppress intermolecular oligomerization and promote intramolecular cyclization. ssDNA circles as small as 20 bases can be prepared. Unfortunately, given the extremely low DNA concentration (far below 1 μm), only very small quantities of ssDNA circles are produced. Variations of the method also have been explored without significant improvement.

In view of the above, there is a longstanding and unmet need for materials and methods for preparing ssDNA circles. It is an object of the present disclosure to provide such materials and methods. This and other objects and advantages, as well as inventive features, will be apparent from the detailed description provided herein.

### SUMMARY

A splint single-stranded DNA (ssDNA), which comprises a linear splint sequence and a hairpin sequence on each side of the linear splint sequence, is provided. The linear splint sequence comprises at least 10 contiguous nucleotides (nts) (such as 10 contiguous nts), half of which hybridize with one end of a ligating linear ssDNA and the other half of which hybridize with the other end of the ligating linear ssDNA. The linear sequence can comprise at least 12 contiguous nts (such as 12 contiguous nts), half of which hybridize with one end of a ligating linear ssDNA and the other half of which hybridize with the other end of the ligating linear ssDNA. The hairpin sequence on one side of the linear splint sequence can differ from the hairpin sequence on the other side of the linear splint sequence. One hairpin sequence can comprise the sequence CTAGTTTTCTAG (SEQ ID NO: 1), and the other hairpin sequence can comprise the sequence GCACTTTTGTGC (SEQ ID NO: 2).

A splint ssDNA-ligating linear ssDNA monomeric complex, which comprises an above-described splint ssDNA, is also provided. The ligating linear ssDNA is at least about 16 nts (such as 16 nts) in length.

Further provided is a method of producing a single-stranded DNA (ssDNA) circle. The method comprises:
(a) incubating an above-described splint ssDNA and a ligating linear ssDNA at least about 16 nts (such as 16 nts) in length under hybridizing conditions, whereupon a splint ssDNA-ligating linear ssDNA monomeric complex is formed, and
(b) incubating the splint ssDNA-ligating linear ssDNA monomeric complex with a DNA ligase, which can seal a nick in DNA, under ligating conditions, whereupon a ssDNA circle is produced. The DNA ligase can be T4 DNA ligase. The concentration of ligating linear ssDNA in (a) can be about 100 μM. The ratio of ligating linear ssDNA to splint ssDNA can be from about 1:1 to about 1:1.5, such as about 1:1.2. The hybridizing conditions comprise annealing in ligation buffer at pH 7-8 and 95-22° C. over about two hours (such as two hours), wherein, when the DNA ligase is T4 DNA ligase, the ligation buffer comprises ATP. The ligating conditions comprise ligating in ligation buffer for at least about 12 to about 16 hours at pH 7-8 and 22-25° C., wherein, when the DNA ligase is T4 DNA ligase, the ligation buffer comprises ATP. The concentration of T4 ligase can be 100 U/μg of ligating linear ssDNA.

Still further provided is a kit comprising an above-described splint ssDNA and instructions for using the splint ssDNA to produce a ssDNA circle. The kit can further comprise components for a ligation buffer, wherein some or all the components can be pre-mixed, and a DNA ligase, wherein, when the DNA ligase is T4 DNA ligase, the kit further comprises ATP, which can be provided separately or combined with the T4 DNA ligase or one or more components of the ligation buffer.

### DESCRIPTION OF THE FIGURES

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings.

FIG. 1 shows enzymatic preparation of single-stranded DNA (ssDNA) circles in accordance with the disclosed method compared to preparation of ssDNA circles in accordance with the conventional method. S6=minimal, conventional splint strand using 2×6=12 nucleotides (nt) in length as example; LN=linear, N-nt long ssDNA; cN=circular, N-nt long ssDNA; (LN)n/(cN)n=oligomers; Hm=new splint strand with a short hairpin at each end of a conventional splint sequence.

FIG. 2 shows secondary structures of LN-splint strand complexes, with (a) showing TGTGCAATAGCT (SEQ ID NO: 10) (S6), complementary strand portion AGCTATTGCACA (SEQ ID NO: 14), and TCAGTCTG (L20); (b) showing CTAGTGTGCAATAGCTCGTG (SEQ ID NO: 15) (H6), complementary strand portion CACGAGCTATTGCACACTAG (SEQ ID NO: 16), and TCAGTCTG (L20); (c) showing CTAGGTGCAATAGCCGTG (SEQ ID NO: 17) (H5), complementary strand portion CACGGCTATTGCACCTAG (SEQ ID NO: 18), and ATCAGTCTGA (SEQ ID NO: 19) (L20); (d) showing CTAGTGCAATAGCGTG (SEQ ID NO: 20) (H4), complementary strand portion CACGCTATTGCACTAG (SEQ ID NO: 21), and CATCAGTCTGAG (SEQ ID NO: 22) (L20); (e) showing SEQ ID NO: 15 (H6), complementary strand portion SEQ ID NO: 16, and L12; (f) showing SEQ ID NO: 15 (H6), complementary strand portion SEQ ID NO: 16, and TG (L14); (g) showing SEQ ID NO: 15 (H6), complementary strand portion SEQ ID NO: 16, and TCTG (L16); (h) showing SEQ ID NO: 15 (H6), complementary strand portion SEQ ID NO: 16, and TCACTG (L18); (i) showing SEQ ID NO: 15 (H6), complementary strand portion SEQ ID NO: 16, and TCAG(t10)TCTG (SEQ ID NO: 23) (L30); and (j) showing SEQ ID NO: 15 (H6), complementary strand portion SEQ ID NO: 16, and and TCAG(t20)TCTG (SEQ ID NO: 24) (L30).

FIG. 3 shows the effect of DNA (L20) concentration on DNA cyclization mediated by H6 (ligation conditions: 16 hours at room temperature).

FIG. 4 shows the effect of ssDNA length on cyclization for L16 and H5 (ligation conditions: 16 hours at room temperature).

While the present disclosure is susceptible to various modifications and alternative forms, exemplary embodiments thereof are shown by way of example in the drawings and are herein described in detail.

### DETAILED DESCRIPTION

For the purposes of promoting an understanding of the principles hereof, reference will now be made to the embodiments illustrated in the drawings and specific language will be used to describe the same. It will nevertheless be understood that no limitation of scope is intended by the description of these embodiments. On the contrary, this disclosure is intended to cover alternatives, modifications, and equivalents as may be included within the spirit and scope of this application as defined by the appended claims.

Small, single-stranded DNA (ssDNA) circles have many applications, such as templating rolling circle amplification (RCA), capturing microRNAs, and scaffolding DNA nanostructures. However, it is challenging to prepare such ssDNA circles, particularly when the DNA size becomes very small, e.g., 20 nucleotides (nt). Often, such short ssDNAs dominantly form concatamers (either linear or circular) due to inter-molecular ligation, instead of forming monomeric ssDNA circles by intramolecular ligation.

In view of the above, the present disclosure is directed to providing a material and a method for overcoming the problem of concatemer formation. ssDNA as short as about 16 nt (e.g., 16 nt) can be enzymatically ligated using a DNA ligase, such as T4 DNA ligase, into monomeric ssDNA circles at a high concentration of template ssDNA (e.g., about 100 μM) with high yield (e.g., about 97%).

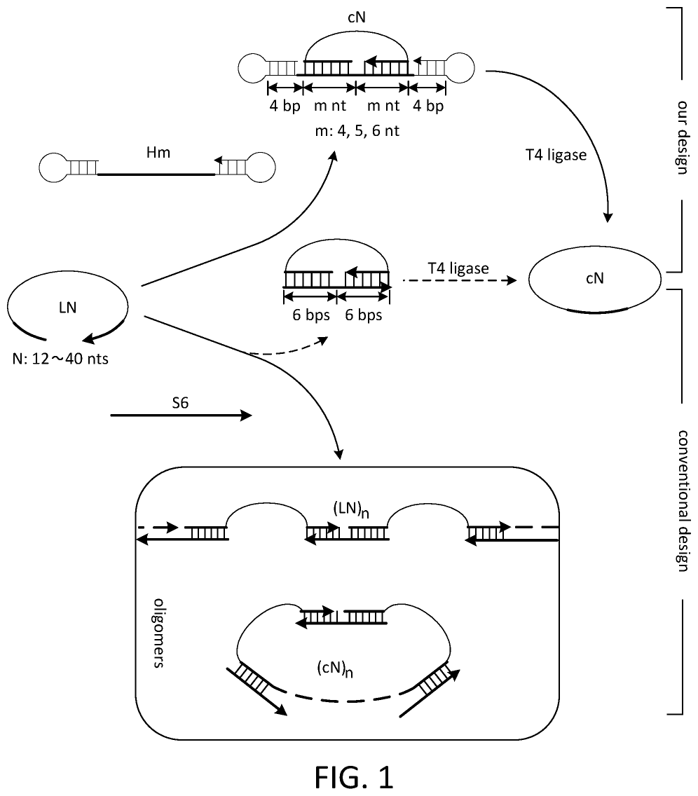

FIG. 1 shows enzymatic preparation of single-stranded DNA (ssDNA) circles in accordance with the disclosed method compared to preparation of ssDNA circles in accordance with the conventional method. S6=minimal, conventional splint strand using 2×6=12 nucleotides (nt) in length as example; LN=linear, N-nt long ssDNA; cN=circular, N-nt long ssDNA; (LN)n/(cN)n=oligomers; Hm=new splint strand with a short hairpin at each end of a conventional splint sequence.

As shown in FIG. 1, an S6 forms a 6-base pair (bp) long duplex with each end of an LN. S6 and LN form either a monomeric complex or oligomers, leading to either cN or (LN)n/(cN)n. Hm form a m-bp-long duplex with each end of LN. The four helical domains in the Hm-LN complex stack onto each other to form a long, pseudo-continuous DNA complex.

Thus, a conventional splint strand hybridizes to the two ends of a target, linear ssDNA to form a pseudo-continuous DNA duplex. Then, T4 DNA ligase covalently joins the ends of the ssDNA to form a circular ssDNA. Each side of the duplex needs to be at least 6 bp long because T4 DNA ligase covers a little more than one helical turn (≥12 pbs) [16]. The shortest splint strand (S6) will be 2×6=12 nts long. In a monomeric complex of an LN and S6, there will be an extra single-stranded loop. When the extra single-stranded loop is short, it introduces too much stress on the monomeric complex, thus favoring alternative oligomeric complexes. High DNA concentrations further promote oligomerization. To promote monomeric DNA circle formation, very low DNA concentrations, such as 0.1 μM, are used. However, such low concentrations decrease enzyme efficiency and are not suitable for large-scale production.

Significant efforts have been developed to improve the ligation, such as placing a hairpin near the joining 5′- and 3′-ends to decrease intermolecular oligomerization [17.] Freezing/drying the dilute DNA solutions to concentrate the DNA to achieve a high concentration of monomeric complexes [9] and step-by-step addition of the linear DNA to allow for better intramolecular cyclization of the ssDNA have improved the ligation process [15]. Another method involves the use of ssDNA CircLigase™[18]. However, this ligase is expensive and has quite a low efficiency, which prevents large-scale synthesis. Furthermore, these methods are only applicable to DNA strands longer than 30 nts.

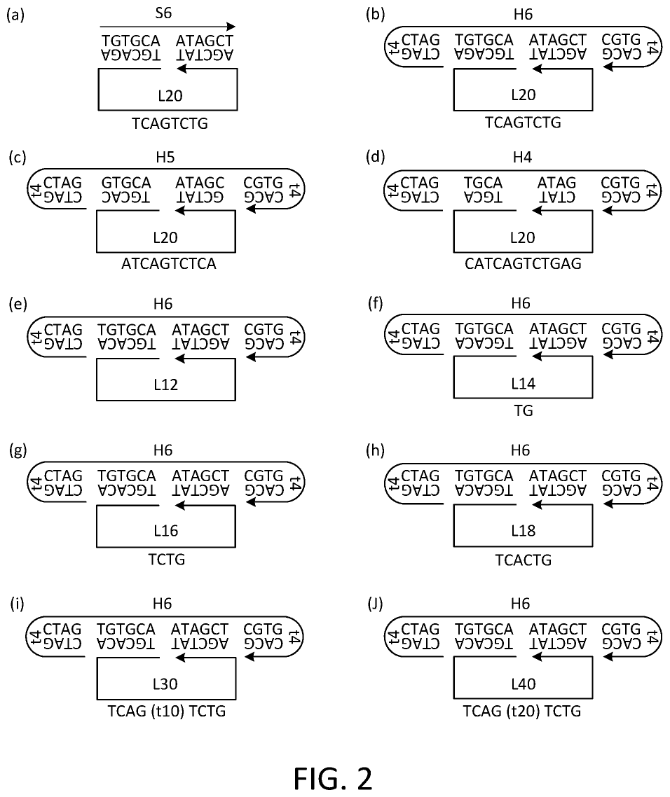

To favor monomeric complex formation and efficient ligation of T4 DNA ligase, a new class of splint strand, Hm (FIGS. 1 and 2), was designed to facilitate LN cyclization. It contains a short hairpin at each end of a splint sequence. Each Hm hybridizes with m bases at either end of the LN. Base stacking between the hairpin stems and LN-Hm duplexes increases the stability of LN-Hm complexes [19]; thus, shorter splint sequences can stably bind to LN strands. The base stacking leads the hairpin stems and the LN-Hm duplexes to form long, pseudo-continuous DNA duplexes to facilitate T4 DNA ligation [17]. Thus, the LN-Hm duplex can be significantly shorter than 6 bps and generate less stress to the red loop region in the monomeric LN-Hm complex. The hairpins add extra intermolecular repulsions, thus favoring intramolecular cyclization.

Thus, provided is a splint single-stranded DNA (ssDNA), which comprises a linear splint sequence and a hairpin sequence on each side of the linear splint sequence. The linear splint sequence comprises at least 10 contiguous nts (such as 10 contiguous nts), half of which hybridize with one end of a ligating linear ssDNA and the other half of which hybridize with the other end of the ligating linear ssDNA. The linear sequence can comprise at least 12 contiguous nts (such as 12 contiguous nts), half of which hybridize with one end of a ligating linear ssDNA and the other half of which hybridize with the other end of the ligating linear ssDNA. The exact sequence of the hairpins can vary as long as the hairpin structures form. The hairpin sequence on one side of the linear splint sequence can differ from the hairpin sequence on the other side of the linear splint sequence. One hairpin sequence can comprise the sequence CTAGTTTTCTAG (SEQ ID NO: 1), and the other hairpin sequence can comprise the sequence GCACTTTTGTGC (SEQ ID NO: 2).

A splint ssDNA-ligating linear ssDNA monomeric complex, which comprises an above-described splint ssDNA, is also provided. The ligating linear ssDNA is at least about 16 nts (such as 16 nts) in length.

Further provided is a method of producing a single-stranded DNA (ssDNA) circle. The method comprises:
(a) incubating an above-described splint ssDNA and a ligating linear ssDNA at least about 16 nts (such as 16 nts) in length under hybridizing conditions, whereupon a splint ssDNA-ligating linear ssDNA monomeric complex is formed, and
(b) incubating the splint ssDNA-ligating linear ssDNA monomeric complex with a DNA ligase, which can seal a nick in DNA, under ligating conditions, whereupon a ssDNA circle is produced. The DNA ligase can be T4 DNA ligase. The concentration of ligating linear ssDNA in (a) can be about 100 μM. The ratio of ligating linear ssDNA to splint ssDNA can be from about 1:1 to about 1:1.5, such as about 1:1.2. The hybridizing conditions comprise annealing in ligation buffer (exemplified herein) at pH 7-8 and 95-22° C. over two hours (such as two hours), wherein, when the DNA ligase is T4 DNA ligase, the ligation buffer comprises ATP. The ligating conditions comprise ligating in ligation buffer for at least about 12 to about 16 hours at pH 7-8 and 22-25° C., wherein, when the DNA ligase is T4 DNA ligase, the ligation buffer comprises ATP. The concentration of T4 ligase can be 100 U/μg of ligating linear ssDNA.

Still further provided is a kit comprising an above-described splint ssDNA and instructions for using the splint ssDNA to produce a ssDNA circle. The kit can further comprise components for a ligation buffer, wherein some or all the components can be pre-mixed, and a DNA ligase, wherein, when the DNA ligase is T4 DNA ligase, the kit further comprises ATP, which can be provided separately or combined with the T4 DNA ligase or one or more components of the ligation buffer.

### EXAMPLES

The following examples serve to illustrate the present disclosure. The examples are not intended to limit the scope of the claimed invention in any way.

### Experimental Methods.

Oligonucleotides. All DNA strands were purchased from IDT, Inc., and used without any treatment. DNA strand sequences are listed below:

L12, 12 nt:
(SEQ ID NO: 3)
pTGCACA AGCTAT
L14, 14 nt:
(SEQ ID NO: 4)
pTGCACAT GAGCTAT
L16, 16 nt:
(SEQ ID NO: 5)
pTGCACATC TGAGCTAT
L18, 18 nt:
(SEQ ID NO: 6)
pTGCACATCA CTGAGCTAT
L20, 20 nt:
(SEQ ID NO: 7)
pTGCACATCAG TCTCAGCTAT
L30, 30 nt:
(SEQ ID NO: 8)
pTGCACATCAG TTTTTTTTTT TCTGAGCTAT
L40, 40 nt:
(SEQ ID NO: 9)
pTGCACATCAG TTTTTTTTTTTTTTTTTTTT TCTGAGCTAT
S6, 12 nt:
(SEQ ID NO: 10)
TGTGCAATAGCT
H6, 36 nt:
(SEQ ID NO: 11)
CTAGttttCTAGTGTGCAATAGCTCGTGttttCACG
H5, 34 nt:
(SEQ ID NO: 12)
CTAGttttCTAG GTGCAATAGC CGTGttttCACG
H4, 32 nt:
(SEQ ID NO: 13)
CTAGttttCTAG TGCAATAG CGTGttttCACG

Lowercase p denotes 5′ phosphorylated. Underlined letters denote the base-pairing regions of the ligating strands with splint strands. Lowercase t denotes the t4 loop. Letters in bold represent the bases flanking the nick point. Letters in italics represent the hairpins.

Ligation. Phosphorylated strands prepared by IDT, Inc., were mixed with the staple strand in a 1:1.2 ratio and annealed from 95 to 22° C. in ligation buffer (50 mM Tris-HCl, 10 mM MgCl₂, 1 mM ATP, 10 mM DTT, pH 7.5 at 25° C., New England BioLabs, Inc). Then, T4 DNA ligase (New England BioLabs, Inc.) was added, and the reaction mixture was incubated at room temperature (22-25° C.) for 16 hours. Always used were 1,000 U of T4 ligase per reaction (100 U/μg of ligating strand).

### Polyacrylamide Gel Electrophoresis.

10% denaturing PAGE gel was prepared with 19:1 acrylamide/bisacrylamide solution, 8 M urea, and TBE buffer containing 89 mM Tris base (pH 8.0), 89 mM boric acid, and 2 mM ethylenediaminetetraacetic acid (EDTA). The gel was run at 55° C. under 600 V on the Hoefer SE 600 electrophoresis system, stained with Stains-All (Sigma), and scanned by an HP scanner (CanoScan LiDe 400).

10% native PAGE gel was prepared with 19:1 acrylamide/bisacrylamide solution and TAE/20 mM Mg²⁺ buffer containing 40 mM Tris base (pH 8.0), 20 mM acetic acid, 2 mM EDTA, and 24 mM magnesium acetate. The gel was run at 4° C. at 220V, stained with Stains-All (Sigma), and scanned by an HP scanner (CanoScan LiDe 400).

Isolation of c20. A 10% denaturing PAGE gel was prepared with 19:1 acrylamide/bisacrylamide solution, 8 M urea, and TBE buffer. The gel was run at 55° C. under 600 V on the Hoefer SE 600 electrophoresis system and then was stained with ethidium bromide (Sigma). The major band was cut under ultraviolet (UV) light and eluted out in 0.1×TBE buffer via electrophoresis and ethanol precipitation. Prior to being purified, the DNA was added to 8 M urea, heated to 95° C. and then snap-cooled before being inserted into the gel wells.

Fetal Bovine Serum Treatment. Purified circular DNA c20 and impure linear ssDNA L20 (see FIG. 2 (c)) were mixed with 10% fetal bovine serum (FBS) and incubated at room temperature (22-25° C.) for 10 minutes, 30 minutes, 1 hour, 2 hours, 4 hours, and 14 hours. These reactions were quenched by mixing an aliquot of the sample with 80% formamide, heating the sample to 95° C. for 3 minutes, and storing on ice until analysis.

### Example 1

A mixture of the ligating strand L20, the splint strand H6, and T4 DNA ligase was incubated. The ligation mixture was then analyzed by denaturing polyacrylamide gel electrophoresis, dPAGE. The experimental data demonstrated exceptionally successful cyclization of L20. The band corresponding to L20 completely disappeared and a fast-moving, new band appeared. The new band was resistant to exonuclease digestion, indicating that it had no free ends. Based on its fast-moving electrophoretic mobility and exonuclease resistance, it was assigned to be a 20-nt-long, circular DNA, c20. The observed mobility was consistent with the mobility reported in the literature [9]. In a control experiment, ligation was conducted with a conventional splint strand, S6, which is complementary to L20 but does not have a hairpin at each end of the splint sequence. Under the identical experimental conditions, no c20 was produced; instead, L20 generated a series of linear and circular oligomers, corresponding to a series of slow-moving bands in the gel.

The near quantitative conversion from a linear DNA into the same-sized, circular DNA is attributed to the unique design of the splint strand. By mixing the splint strand and the ssDNA, they could form a 1:1 complex or oligomeric complexes. For oligomers, hairpins from adjacent splint strands are in close proximity and strongly, electrostatically repel each other, thus decreasing the stability of the oligomeric complexes. In a monomeric complex of L20-H6, such repulsion is avoided. Thus, the monomeric complex is preferentially formed over oligomers. After incubation of L20 and H6, the DNA sample was analyzed by native PAGE, nPAGE. Besides the bands of L20 and H6, a new, dominant band appeared, indicating the formation of a stable L20-H6 (1:1) complex. This monomeric complex ensures intra-complex ligation to generate the monomeric circle, c20. In contrast, L20 and S6 cannot form any stable complex; and after ligating these molecules, L20 would, linearly or circularly, oligomerize. Ligation efficiency was also a function of the temperature and duration. Ligation reached ˜75% efficiency after incubation for 2 hours at 22° C., and nearly completed (˜90%) after 16 hours.

### Example 2

To test whether the effective splint sequence can be shortened with additional hairpins, another two splint strands, H5 and H4, which base pair with 5 bases and 4 bases, respectively, at each end of the linear, ligating DNA, were prepared (FIGS. 1 and 2(c) and (d)). By the dPAGE, L20 is nearly quantitatively converted into c20 with H5. However, with H4, the ligation efficiency significantly decreased and some L20 remained. Furthermore, one band with slightly slower mobility than L20, corresponding to 5′-adenylpyrophosphorylated L20 (5′ AppL20), appeared. The 5′ AppDNA strand is an intermediate of DNA ligation and will accumulate if the following step (cyclization) cannot efficiently be processed [20,21]. These data are also supported by the nPAGE data. L20-H5 formed a stable complex, although some dissociation exists as evidenced by a smear shown below the complex band. The L20-H4 complex is much less stable and constantly dissociates during electrophoresis, leading to a darker smear below the complex band and a strong H4 band. From the analysis of the ligation and complex formation, stable, monomeric linear DNA-splint complexes promote an efficient conversion of the linear DNA into the desired, monomeric circular DNA.

### Example 3

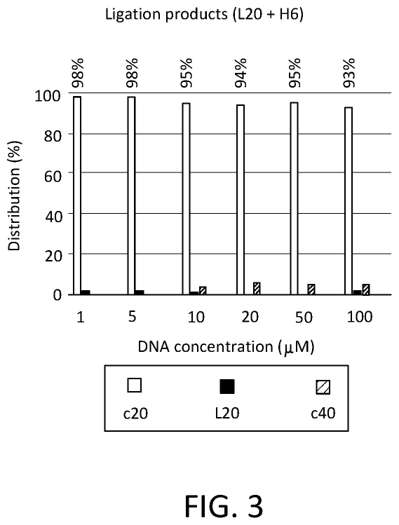

Oligomerization is an unwanted, competing process to cyclization and becomes even worse at increased DNA concentrations. To examine whether this problem can be overcome, ligation at a series of different DNA concentrations was conducted, and the cyclization efficiency was estimated based on the band intensities with Image J, an image processing software [22]. Impressively, L20 nearly quantitatively cyclized into c20 in the DNA concentration range of 1-100 μM when H6 and H5 were used as splint strands (FIG. 3). In sharp contrast, when the conventional splint strand S6 was used at DNA concentration of 20 μM, the c20 band was not observed in the gel. Instead, a series of bands corresponding to L20 oligomers appeared. Consistent with previous experiments, H4 failed to promote efficient ligation and a significant amount (˜40%) of L20 strands remained as the ligation intermediate 5′ AppL20.

### Example 4

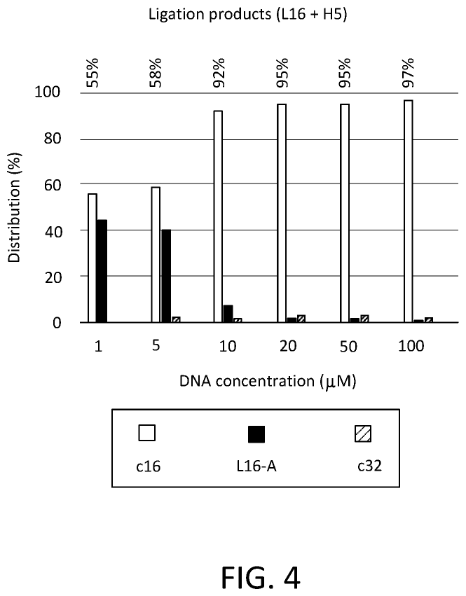

To test whether DNA strands shorter than 20 nts could by cyclized, four DNA strands, L18, L16, L14 and L12, which are 18, 16, 14, and 12 nts long, respectively, were ligated (FIG. 4). Through the experimental data shown, DNA strands as short as 16 nts long were nearly quantitatively cyclized at the DNA concentration of 100 μM (FIG. 4). It is worth noting that the overall ligation yield increases as the DNA concentration increases. The ligation yield is only 55% when the DNA concentration is 1 μM but reaches 97% when the DNA concentration is 100 μM, presumably due to the increased ligase concentration. The molar ratio of DNA and ligase was the same for all experiments. Increased reagent concentrations led to faster ligation kinetics. When the DNA length was further decreased to 14 nts long, no c14 was observed; and instead, L14 oligomerized into longer DNA strands first and then cyclized. The dominant product was c28, the dimeric circle of L14. A similar situation occurred with L12. Thus, ssDNA circles as small as 16 nts long, but not shorter, can be nearly quantitatively produced.

On the other hand, longer DNA strands, for example L30 and L40, which are 30 and 40 nts long, respectively, can be cyclized (FIG. 4). Both strands were quantitatively converted from linear ssDNAs into circles. After ligation, the original bands corresponding to L30 and L40 completely disappeared. Instead, two new exonuclease-resistant bands showed up, corresponding to the monomeric circles c30 and c40.

### Example 5

To demonstrate the stability of circular DNAs, c20 and L20 were incubated with 10% fetal bovine serum (FBS) for 14 hours. While c20 had no obvious degradation, 120 started to degrade after 1 hour and completely degraded after 14 hours. Thus, the circular DNAs are indeed more stable than linear DNAs in biological fluids.

### REFERENCES

1. Gleave, Martin E.; Monia, Brett P. Antisense therapy for cancer. Nat. Rev. Canc. 5, 468-479 (2005).
2. Li, Jinbo; Tan, Subee; Kooger, Romain; Zhang, Chenyu; Zhang, Yan. MicroRNAs as novel biological targets for detection and regulation. Chem. Soc. Rev. 43, 506-517 (2014).
3. Deng, Rujie; Tang, Longhua; Tian, Qianqian; Wang, Ying; Lin, Lei; Li, Jinghong. Toehold-initiated Rolling Circle Amplification for Visualizing Individual MicroRNAs In Situ in Single Cells. Angew. Chem. Intl. Ed. 53, 2389-2393 (2014).
4. Li, Qian; Wang, Dong; Yuan, Ming; He, Bingfeng; Li, Jin; Ma, Chengde; Wang, Guansong; Qian, Hang. Capturing intracellular oncogenic microRNAs with self-assembled DNA nanostructures for microRNA-based cancer therapy. Chem. Sci. 9, 7562-7568 (2018).
5. Keefe, Anthny D.; Pai, Supriya; Ellington, Andrew. Aptamers as therapeutics. Nat. Rev. Drug Discov. 9, 537-550 (2010).
6. Tan, Weihong; Donovan, Michael J.; Jiang, Jianhui. Aptamers from Cell-Based Selection for Bioanalytical Applications. Chem. Rev. 113, 2842-2862 (2013).
7. Zhou, Wenhu; Saran, Runjhun; Liu, Juewen. Metal Sensing by DNA. Chem. Rev. 117, 8272-8325 (2017).
8. Zuo, Xialei; Song, Shiping; Zhang, Jiong; Pan, Dun; Wang, Lihua; Fan, Chunhai. A Target-Responsive Electrochemical Aptamer Switch (TREAS) for Reagentless Detection of Nanomolar ATP. J. Am. Soc. Chem. 129, 1042-1043 (2007).
9. Gubu, Amu; Wang, Jing; Jin, Hongwei; Tang, Xinjing. Synthesis and “DNA Interlocks” Formation of Small Circular Oligodeoxynucleotides. ACS Appl. Mat. Int. 12, 12584-12590 (2020).
10. Kool, Eric T. Circular oligonucleotides: new concepts in oligonucleotide design. Biophys. 25, 1-28 (1996).
11. Nallur, Girish; Luo, Chenghua; Fang, Linhua; Cooley, Stephanie; Dave, Varshal; Lambert, Jeremy; Kukanskis, Kari; Kingsmore, Stephen; Lasken, Roger; Schweitzer, Barry. Signal amplification by rolling circle amplification on DNA microarrays. Nucleic Acids Res. 29, e118 (2001).
12. Zhou, Yuntao; Huang, Qing; Gao, Jimin; Lu, Jianxin; Shen, Xizhong; Fan, Chunfai. A dumbbell probe-mediated rolling circle amplification strategy for highly sensitive microRNA detection. Nucleic Acids Res. 38, e156 (2010).
13. Li, Jing; Lin, Li; Yu, Jiantao; Zhai, Shiyao; Liu, Guoyuan; Tian, Leilei. Fabrication and Biomedical Applications of “Polymer-Like” Nucleic Acids Enzymatically Produced by Rolling Circle Amplification. ACS Appl. Bio. Mater. 2, 4106-4120 (2019).
14. Sakhabutdinova, Assol R.; Maksimova, Mariia A.; Garafutdinov, Ravilf R. Synthesis of Circular DNA Templates with T4 RNA Ligase for Rolling Circle Amplification. Mol. Biol. 51, 639-646 (2017).
15. An, Ra; Li, Qi; Fan, Yiqiao; Pan, Xiaoming; Komiyama, Makoto; Liang, Xingguo. Highly efficient preparation of single-stranded DNA rings by T4 ligase at abnormally low Mg(II) concentration. Nucleic Acids Res. 45, e139 (2017).
16. Shi, Ke; Bohl, Thomas E.; Park, Jeonghyun; Zasada, Andrew; Malik, Shray; Banerjee, Surajit; Tran, Vincent; Li, Na; Yin, Zhiqi; Kurniawan, Fredy; Orellana, Kayo; Aihara, Hideki. T4 DNA ligase structure reveals a prototypical ATP-dependent ligase with a unique mode of sliding clamp interaction. Nucleic Acid Res. 46, 10474-10488 (2018)
17. Cui, Yixiao; Han, Xutiange; An, Ran; Zhang, Yaping; Cheng, Kai; Liang, Xingguo; Komiyama, Makoto. Terminal hairpin in oligonucleotide dominantly prioritizes intramolecular cyclization by T4 ligase over intermolecular polymerization: an exclusive methodology for producing ssDNA rings. Nucleic Acids Res. 46, e132 (2018).
18. Li, Qingting; Zhang, Shu; Li, Wei; Ge, Zhilei; Fan, Chunhai; Gu, Hongzhou. Programming CircLigase Catalysis for DNA Rings and Topologies. Anal. Chem. 93, 1801-1810 (2021).
19. Yakovchuk, Peter; Protozanova, Ekaterina; Frank-Kamenetskii, Maxim D. Base-Stacking and Base-Pairing Contributions into Thermal Stability of The DNA Double Helix. Nucleic Acids Res. 34, 564-574 (2006).
20. Cherepanov, Alexey V.; de Vries, Simon. Kinetics and thermodynamics of nick sealing by T4 DNA ligase. Eur. J. Biochem. 270, 4315-4326 (2003).
21. Wang, Yangming; Silverman, Scott K. Efficient RNA 5′-adenylation by T4 DNA ligase to facilitate practical applications. RNA. 12, 1142-1146 (2006).
22. Schneider, C. A.; Rasband, W. S.; Eliceiri, K. W. NIH Image to ImageJ: 25 years of image analysis. Nat. Methods. 9, 671-675 (2012).
23. Chandrasekaran, Arun R.; Punnoose, Jibin A.; Zhou, Lifeng; Dey, Paromita; Dey, Bijan K; Halvorsen, Ken. DNA nanotechnology approaches for microRNA detection and diagnosis. Nucleic Acids Res. 47, 10489-10505 (2019).
24. Wu, L.; Wang, Y.; Wu, J.; Lv, C.; Wang, J.; Tang, X. Caged Circular Antisense Oligonucleotides for Photomodulation of RNA Digestion and Gene Expression in Cells. Nucleic Acids Res. 41, 677-686 (2013).
25. Zheng, Hongning; Xiao, Minyu; Yan, Qin; Ma, Yinzhou; Xiao, Shou-Jun. Small Circular DNA Molecules Act as Rigid Motifs to Build DNA Nanotubes. J. Am. Chem. Soc. 136, 10194-10197 (2014).
26. Kool, Eric T. Recognition of DNA, RNA, and Proteins by Circular Oligonucleotides. Acc. Chem. Res. 31, 502-510 (1998).
27. Seeman, Nadrian C. From Genes to Machines: DNA Nanomechanical Devices. Trends Biochem. Sci. 30, 119-125 (2012).
28. Wezmann, Yossi; Braunscheig, Adam B.; Wilner, Ofer i.; Cheglakov, Zoya; Willner, Itamer. A polycatenated DNA scaffold for the one-step assembly of hierarchical nanostructures. Proc. Natl. Acad. Sci. USA. 105, 5289-5294 (2008).
29. Shen, Tingting; Zhang, Yu; Lei, Lei; Zhang, Xiao-Bing; Zhu, Guizhi. Single-stranded circular DNA theranostics. Theranostics. 12, 35-47 (2022).
30. Qingting Li, Qingting Li, Shu Zhang, Wei Li, Zhilei Ge, Chunhai Fan, and Hongzhou Gu. Programming CircLigase Catalysis for DNA Rings and Topologies. Anal. Chem. 93, 1801-1810 (2021).
31. Gang Chen, Di Liu, Chunbai He, Theodore R. Gannett, Wenbin Lin, and Yossi Weizmann. Enzymatic Synthesis of Periodic DNA Nanoribbons for Intracellular pH Sensing and Gene Silencing. J. Am. Chem. Soc. 137, 3844-3851 (2015).
32. Soong Ho Um 1, Jong Bum Lee, Nokyoung Park, Sang Yeon Kwon, Christopher C. Umbach, Dan Luo. Enzyme-catalysed assembly of DNA hydrogel. Nat. Mater. 5, 797-801 (2006).
33. Jaepil Jeong, Hyejin Kim, Dong Jun Lee, Byung Jun Jung, and Jong Bum Lee. RCA-Based Biosensor for Electrical and Colorimetric Detection of Pathogen DNA. Nanoscale Res Lett 11, 242 (2016).
34. So Yeon Ahn, Jeonghun Kim, Srivithya Vellampatti, Sung Oh, Yong Taik Lim, Sung Ha Park, Dan Luo, Junho Chung, and Soong Ho Um. Protein-Encoding Free-Standing RNA Hydrogel for Sub-Compartmentalized Translation. Adv. Mater. 34, 2110424 (2022).

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference. In the event of inconsistent usages between this document and those documents so incorporated by reference, the usage in the incorporated reference should be considered supplementary to that of this document; for irreconcilable inconsistencies, the usage in this document controls.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure. The term “or” is used to refer to a nonexclusive “or” unless otherwise indicated.

Unless defined otherwise, all technical and scientific terms used herein have the same meaning as commonly understood to one of ordinary skill in the art. The following terms and phrases shall have the meaning indicated.

The term “about,” when referring to a number or a numerical value or range (including, for example, whole numbers, fractions, and percentages), means that the number or numerical range referred to is an approximation within experimental variability (or within statistical experimental error) and thus the numerical value or range can vary between 1% and 15% of the stated number or numerical range (e.g., +/−5% to 15% of the recited value, such as within 10%, within 5%, or within 1% of a stated value or stated limit of a range) provided that one of ordinary skill in the art would consider equivalent to the recited value (e.g., having the same function or result). The term “substantially” can allow for a degree of variability in a value or range, for example, within 90%, within 95%, 99%, 99.5%, 99.9%, 99.99%, or at least about 99.999% or more of a stated value or of a stated limit of a range.

In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section.

What is claimed is:

1. A splint single-stranded DNA (ssDNA), which comprises a linear splint sequence and a hairpin sequence on each side of the linear splint sequence.

2. The splint ssDNA of claim 1, wherein the linear splint sequence comprises at least 10 contiguous nucleotides (nts), half of which hybridize with one end of a ligating linear ssDNA and the other half of which hybridize with the other end of the ligating linear ssDNA.

3. The splint ssDNA of claim 1, wherein the linear splint sequence comprises at least 12 contiguous nts, half of which hybridize with one end of a ligating linear ssDNA and the other half of which hybridize with the other end of the ligating linear ssDNA.

4. The splint ssDNA of claim 1, wherein the hairpin sequence on one side of the linear splint sequence differs from the hairpin sequence on the other side of the linear splint sequence.

5. The splint ssDNA of claim 4, wherein one hairpin sequence comprises the sequence CTAGTTTTCTAG (SEQ ID NO: 1) and the other hairpin sequence comprises the sequence GCACTITTGTGC (SEQ ID NO: 2).

6. A splint single-stranded DNA (ssDNA)-ligating linear ssDNA monomeric complex, which comprises the splint ssDNA of claim 1.

7. The splint ssDNA-ligating linear ssDNA monomeric complex of claim 6, wherein the linear splint sequence comprises at least 10 contiguous nucleotides (nts), half of which hybridize with one end of a ligating linear ssDNA and the other half of which hybridize with the other end of the ligating linear ssDNA.

8. The splint ssDNA-ligating linear ssDNA monomeric complex of claim 6, wherein the linear splint sequence comprises at least 12 contiguous nts, half of which hybridize with one end of a ligating linear ssDNA and the other half of which hybridize with the other end of the ligating linear ssDNA.

9. The splint ssDNA-ligating linear ssDNA monomeric complex of claim 6, wherein the hairpin sequence on one side of the linear splint sequence differs from the hairpin sequence on the other side of the linear splint sequence.

10. The splint ssDNA-ligating linear ssDNA monomeric complex of claim 6, wherein one hairpin sequence comprises the sequence CTAGTTTTCTAG (SEQ ID NO: 1) and the other hairpin sequence comprises the sequence GCACTTTTGTGC (SEQ ID NO: 2).

11. The splint ssDNA-ligating linear ssDNA monomeric complex of claim 6, wherein the ligating linear ssDNA is at least about 16 nucleotides (nts) in length.

12. A method of producing a single-stranded DNA (ssDNA) circle, which method comprises:
(a) incubating a splint ssDNA of claim 1 and a ligating linear ssDNA at least about 16 nts in length under hybridizing conditions, whereupon a splint ssDNA-ligating linear ssDNA monomeric complex is formed, and
(b) incubating the splint ssDNA-ligating linear ssDNA monomeric complex with a DNA ligase, which can seal a nick in DNA, under ligating conditions, whereupon a ssDNA circle is produced.

13. The method of claim 12, wherein the DNA ligase is T4 DNA ligase.

14. The method of claim 12, wherein the concentration of ligating linear ssDNA in (a) is about 100 μM.

15. The method of 12, wherein the ratio of ligating linear ssDNA to splint ssDNA is from about 1:1 to about 1:1.5.

16. The method of claim 15, wherein the ratio of ligating linear ssDNA to splint ssDNA is about 1:1.2.

17. The method of claim 12, wherein the hybridizing conditions comprise annealing in ligation buffer at pH 7-8 and 95-22° C. over about two hours, wherein, when the DNA ligase is T4 DNA ligase, the ligation buffer comprises ATP.

18. The method of claim 12, wherein the ligating conditions comprise ligating in ligation buffer for at least about 12 to about 16 hours at pH 7-8 and 22-25° C., wherein, when the DNA ligase is T4 DNA ligase, the ligation buffer comprises ATP.

19. The method of claim 18, wherein the concentration of T4 ligase is 100 U/μg of ligating linear ssDNA.

20. A kit comprising the splint ssDNA of claim 1 and instructions for using the splint ssDNA to produce a ssDNA circle.

21. The kit of claim 20, which further comprises components for a ligation buffer, wherein some or all of the components can be pre-mixed, and a DNA ligase, wherein, when the DNA ligase is T4 DNA ligase, the kit further comprises ATP, which can be provided separately or combined with the T4 DNA ligase or one or more components of the ligation buffer.


SEQUENCE LISTING (24 sequences)

SEQ ID NO: 1 - DNA, 12 bp, synthetic construct
  source 1..12: mol_type = other DNA; note = hairpin sequence
ctagttttctag

SEQ ID NO: 2 - DNA, 12 bp, synthetic construct
  source 1..12: mol_type = other DNA; note = hairpin sequence
gcacttttgtgc

SEQ ID NO: 3 - DNA, 12 bp, synthetic construct
  source 1..12: mol_type = other DNA; note = LT12
tgcacaagctat

SEQ ID NO: 4 - DNA, 14 bp, synthetic construct
  source 1..14: mol_type = other DNA; note = L14
tgcacatgagctat

SEQ ID NO: 5 - DNA, 16 bp, synthetic construct
  source 1..16: mol_type = other DNA; note = L16
tgcacatctgagctat

SEQ ID NO: 6 - DNA, 18 bp, synthetic construct
  source 1..18: mol_type = other DNA; note = L18
tgcacatcactgagctat

SEQ ID NO: 7 - DNA, 20 bp, synthetic construct
  source 1..20: mol_type = other DNA; note = L20
tgcacatcagtctgagctat

SEQ ID NO: 8 - DNA, 30 bp, synthetic construct
  source 1..30: mol_type = other DNA; note = L30
tgcacatcagtttttttttttctgagctat

SEQ ID NO: 9 - DNA, 40 bp, synthetic construct
  source 1..40: mol_type = other DNA; note = L40
tgcacatcagtttttttttttttttttttttctgagctat

SEQ ID NO: 10 - DNA, 12 bp, synthetic construct
  source 1..12: mol_type = other DNA; note = S6
tgtgcaatagct

SEQ ID NO: 11 - DNA, 36 bp, synthetic construct
  source 1..36: mol_type = other DNA; note = H6
ctagttttctagtgtgcaatagctcgtgttttcacg

SEQ ID NO: 12 - DNA, 34 bp, synthetic construct
  source 1..34: mol_type = other DNA; note = H5
ctagttttctaggtgcaatagccgtgttttcacg

SEQ ID NO: 13 - DNA, 32 bp, synthetic construct
  source 1..32: mol_type = other DNA; note = H4
ctagttttctagtgcaatagcgtgttttcacg

SEQ ID NO: 14 - DNA, 12 bp, synthetic construct
  source 1..12: mol_type = other DNA; note = complementary strand portion to SEQ ID NO: 10
agctattgcaca

SEQ ID NO: 15 - DNA, 20 bp, synthetic construct
  source 1..20: mol_type = other DNA; note = H6
ctagtgtgcaatagctcgtg

SEQ ID NO: 16 - DNA, 20 bp, synthetic construct
  source 1..20: mol_type = other DNA; note = complementary strand portion to SEQ ID NO: 15
cacgagctattgcacactag

SEQ ID NO: 17 - DNA, 18 bp, synthetic construct
  source 1..18: mol_type = other DNA; note = H5
ctaggtgcaatagccgtg

SEQ ID NO: 18 - DNA, 18 bp, synthetic construct
  source 1..18: mol_type = other DNA; note = complementary strand portion to SEQ ID NO: 17
cacggctattgcacctag

SEQ ID NO: 19 - DNA, 10 bp, synthetic construct
  source 1..10: mol_type = other DNA; note = L20
atcagtctga

SEQ ID NO: 20 - DNA, 16 bp, synthetic construct
  source 1..16: mol_type = other DNA; note = H4
ctagtgcaatagcgtg

SEQ ID NO: 21 - DNA, 16 bp, synthetic construct
  source 1..16: mol_type = other DNA; note = complementary strand portion to SEQ ID NO: 20
cacgctattgcactag

SEQ ID NO: 22 - DNA, 12 bp, synthetic construct
  source 1..12: mol_type = other DNA; note = L20
catcagtctgag

SEQ ID NO: 23 - DNA, 18 bp, synthetic construct
  source 1..18: mol_type = other DNA; note = L30
tcagtttttttttttctg

SEQ ID NO: 24 - DNA, 28 bp, synthetic construct
  source 1..28: mol_type = other DNA; note = L40
tcagtttttttttttttttttttttctg